(19) United States
(12) Patent Application Publication
(10) Pub. No.: US 2025/0059529 A1
(43) Pub. Date: Feb. 20, 2025

(54) RNA/DNA ISOLATION KIT WITH HIGH EFFICIENCY

(71) Applicant: BAHCESEHIR UNIVERSITESI, Besiktas/Istanbul (TR)

(72) Inventors: Nejla Lorin DOGANTURK, Istanbul (TR); Sedef OZUNLU, Istanbul (TR); Ozan AKDOGAN, Istanbul (TR)

(73) Assignee: BAHCESEHIR UNIVERSITESI, Besiktas/Istanbul (TR)

(21) Appl. No.: 18/722,682

(22) PCT Filed: Dec. 19, 2022
(86) PCT No.: PCT/TR2022/051527

§ 371 Date: Jun. 21, 2024

(30) Foreign Application Priority Data
Dec. 21, 2021 (TR) 2021/020554

(51) Int. Cl. C12N 15/10

(52) U.S. Cl. CPC C12N 15/1013

(57) ABSTRACT

The present invention relates to a magnetic gradient-based nucleic acid isolation kit that provides nucleic acid (RNA/DNA) isolation from biological materials. The kit comprises a flat-bottomed tube (1) made of glass or its derivatives, configured to contain biological material: a modular and reusable magnetic gradient patch (2) that is made of ferromagnetic metal powder and polymer, that is positioned outside and below the tube such that it is not in direct contact with the biological material to be placed in the tube (1), that is in direct contact with the entire flat bottom of the tube (1); and a mold (3) positioned under the flat-bottom tube (1), configured such that the magnetic gradient patch (1) is put into it. By means of the present invention, a kit is provided for target nucleic acid retention that does not require the direct binding of magnetic elements to nucleic acids and their cleaning, that has a magnetic gradient patch that can be used repeatedly, that accelerates and facilitates the nucleic acid isolation step, thereby accelerating the process in studies that require the use of nucleic acids.

### TECHNICAL FIELD OF THE INVENTION

The present invention relates to a magnetic gradient-based nucleic acid isolation kit that provides nucleic acid (RNA/DNA) isolation from biological materials.

### STATE OF THE ART

DNA isolation is the process of obtaining DNA by removing it from the cell, environment, or virus it normally resides in, and is often the first step in methods used to detect bacteria and viruses in the environment and diagnose disease and genetic disorders. Isolated or purified DNA is then used in many studies such as polymerase chain reaction (PCR), Southern blot, DNA sequencing and transfection. RNA isolation is the process of obtaining RNA from biological samples, and is a difficult and long-term procedure due to the presence of ribonuclease enzyme, which can rapidly degrade the RNA present in cells and tissues. Isolated or purified RNA is subsequently used in many studies such as cloning, reverse transcription (RT) for cDNA synthesis, RT-PCR, RT-qPCR, and RNA-sequencing.

Nucleic acid isolation is a multi-step method, and if it is not performed manually, it is a method that requires costly devices. On the other hand, if RNA or DNA isolation is performed manually, it reveals the need for many different steps and chemical materials. In addition, chemicals used in the isolation of nucleic acids cannot be reused since they are in direct contact with the target nucleic acid. The fact that the elements in the kits are not reusable also causes the isolation processes to be high cost. This multi-step isolation method includes multiple pipetting steps and inter-tube transfer steps. The risk of contamination of the sample used during these steps also increases.

In general, nucleic acid isolation consists of lysis, purification, and precipitation steps. However, the chemicals used for RNA isolation and the chemicals used for DNA isolation differ. RNA and DNA isolation is also a disadvantage for experiments performed after isolation especially due to the long duration of the isolation phase, the fact that it includes many process steps, the sample used is contaminated or degraded. The negativities experienced during the isolation phase also negatively affect the process of subsequent studies such as disease detection, sequencing, in vitro experiments, in which the isolated RNA or DNA will be used.

For example, PCR method, which requires RNA isolation for diagnosis today and is actively used in the diagnosis of Covid-19, poses a disadvantage for the current pandemic situation due to the fact that the time to reach the result is quite long and the amount of possible contamination increases during this time. As a solution thereof, developing an isolation method that provides results in a short time and facilitates RNA isolation could play an important role in diagnosing potentially infected people in a short time and preventing the spread of the epidemic. There are many RNA and DNA isolation kits released by different companies and universities, and most of them are actively used and sold. However, the cost of these kits is high and therefore their availability is not equally easy for everyone. Apart from the cost, the methods in the prior art are disadvantageous since they require the use of many chemicals and include many process steps.

In the prior art, magnetic extraction-based methods are used for the isolation/extraction of nucleic acids. Today, nucleic acids are isolated by spin column or magnetic bead extraction by using sample and application-specific kits. Magnetic bead extraction requires a lysis buffer to lyse cells and, depending on the sample, a physical lysis method. After sample lysis, magnetic beads are added that bind the nucleic acids of the samples. The tubes are then placed on a magnet and the supernatant aspirated to remove undesired unbound material. This step is repeated several times, changing the wash buffer in between, and for the final phase, an elution buffer is added to separate the nucleic acids from the beads before transferring the samples to a different vessel. Processes in which isolation is provided by magnetic methods generally require process steps such as using chemicals, binding nucleic acids to magnetic beads, and subsequent washing of magnetic beads from nucleic acid. The isolation results in a long time and is costly due to the necessity of specifically binding the DNA or RNA to the magnetic carrier in these process steps. These multi-step processes involve many costly aspects, including the synthesis of magnetic beads and the application of specific modifications to the synthesized beads as magnetic carriers. In addition, the attractiveness of the magnetic beads is insufficient to achieve bonding without any chemicals.

The patent document numbered U.S. Pat. No. 11,059,050B2 in the prior art discloses a method developed for magnetic capture of target molecules (e.g., microbes) in a fluid. Here, it is aimed to magnetically capture of a target molecule (e.g., cells, microbes, small molecules, chemicals, drugs, proteins, and/or nucleic acids) from a fluid, including bodily fluids such as blood, food, water, and environmental sources. For this, in the patent document numbered U.S. Pat. No. 11,059,050B2 it was developed a device and method that combines the target molecule with the liquid in which it is located and contains specially modified magnetic particles to bind to the target molecule. After determining the target molecule in the liquid, the magnetic nanoparticles to be combined with the liquid are modified specifically for the molecule. This liquid-nanoparticle mixture is then transferred to the magnetic separation chamber, and it is ensured that the particles that have carried out molecule retention are captured by the magnetic field. However, the preliminary preparation steps used in said method and the process steps applied before/after the retention prolong the isolation process. In addition, nanoparticle synthesis and chemical modification are performed during the preparation of the modified magnetic nanoparticle used here, that is, long-term and costly preliminary preparation processes are applied for target molecule retention.

In the patent application numbered EP1260595A2 in the prior art discloses a method for nucleic acid isolation by utilizing a nucleic acid-bound magnetic carrier to extract or purify nucleic acid from a biological material. Here, iron oxide particles synthesized to bond with nucleic acid and coated with silica, the sample to be isolated and the isolation solution are collected in the centrifuge tube and the particles that performs retention are separated with the help of a magnetic stand. In the document numbered EP1260595A2, the nucleic acid isolation solution is placed in a centrifuge tube and then a whole blood sample is added to the solution and mixed. Magnetic silica particles in sterilized water are added to the tube. The sample in the tube is mixed repeatedly and bond formation between the particles and nucleic acids is ensured. Particles are separated by a tube positioned on a magnetic stand, and then the sample is washed multiple times with washing solutions and alcohol to dissolve the particle-nucleic acid bond. However, the long washing steps after the preliminary preparation and bonding used in the method prolong the isolation process. In addition, methods that take a long time, involve a large number of processing steps and are costly are applied for the preparation of silica-coated iron nanoparticles used here and for cleaning these particles from samples after binding of the nanoparticles to nucleic acids.

Studies that require the use of isolated nucleic acids also have disadvantages that result in a long time and complications in the process due to fact that the isolation methods and kits in the state of the art require long and costly preliminary preparations for target nucleic acid retention, there are many process steps in the kits, and the risk of contamination of the samples is high, the magnetic carriers used in the kits specifically bind to nucleic acids and the magnetic carriers need to be cleaned from nucleic acids after nucleic acid retention occurs, isolation processes take long and include complex steps. In addition, chemicals and magnetic elements in the kits of the previous art cannot be used repeatedly since they are in direct contact with the samples, and this creates a cost disadvantage.

There is a need to develop methods and kits that do not require long-term and costly preliminary preparation steps for target nucleic acid retention, that comprises a small number of process steps, have a low risk of contamination, that do not require direct binding and cleaning of magnetic elements to nucleic acids, that have elements that can be used over and over again, that accelerate and facilitate the nucleic acid isolation step and thereby accelerating the process and provide advantages in studies that require the use of nucleic acids due to the above mentioned disadvantages.

### BRIEF DESCRIPTION OF THE INVENTION

The present invention discloses a magnetic gradient-based nucleic acid isolation kit that provides nucleic acid (RNA/DNA) isolation from biological materials. Said kit comprises a modular and reusable magnetic gradient patch, a mold in which this gradient patch is put, and a tube with a flat bottom, made of glass or its derivatives, positioned on the pattern with said patch. The magnetic gradient patch included in the kit, which is the subject of the present invention, provides nucleic acid retention from biological samples.

The first object of the present invention is to provide a kit that provides nucleic acid isolation from biological materials, e.g body fluid, in a short time compared to the prior art without the need for preliminary preparation steps and long separation/washing steps after bonding with the magnetic element. In the present invention, magnetic gradient patch positioned outside and below the flat bottom glass tube so as not to come into contact with the product interacts magnetically with the sample placed in the tube. By means of the flat bottom of the tube in the kit, the surface area where the magnetic gradient patch contacts the tube is expanded. The entire flat bottom of the tube is in direct contact with the magnetic gradient patch. Thus, the magnetic gradient effect area applied by the magnetic gradient patch to the biological material is expanded and the applied attraction force is increased. The interaction of the biological material with the patch and the amount of nucleic acids in the biological material being affected by the magnetic gradient/attracted towards the magnetic gradient are increased by means of the fact that the bottom is thinner compared to the prior art, in addition to the flat bottom of said tube; thereby achieving nucleic acid retention with high affinity provided without the need for any chemicals. Nucleic acid retention is ensured by the magnetic patch at the base of the glass tube, and the nucleic acids on the glass tube base are easily collected by removing the modular base.

Another object of the present invention is to provide nucleic acid isolation with high magnetic attraction without the need for any chemicals. The magnetic capturing effect/magnetic attraction power of the magnetic gradient structures is much higher than the magnetic beads found in the prior art due to the presence of metals in powder form in magnetic gradient structures, the decrease in the surface area of metal powders, and the increase in the distribution of metal powders in the structure. While preparing the magnetic gradient patch, which is the subject of the present invention, magnetic gradients are increased by using powdered ferromagnetic metals. The magnetic gradients present in the metal powder are increased by means of adding ferromagnetic metal powder into the polymer and homogeneously dispersing it in the polymer. The gradient increases since the surface area of the metal powders trapped in the polymer decreases and its distribution/dispersion within the polymer increases.

Isolation is easily achieved without the application of process steps such as binding or cleaning the nucleic acids to the magnetic element since the sample used with the present invention is not in direct contact with the magnetic gradient patch. In addition, the metal powders used in the present invention are easily prepared with a ball milling device, and used in the kit of the present invention without the need for an additional chemical modification or preparation step. In the present invention, a patch is prepared by pouring polymer material or epoxy resin on metal powder, thus, a kit that is both easy to manufacture and easy to apply is provided with the present invention.

Another object of the present invention is to reduce the risk of contamination by reducing the number of process steps in nucleic acid isolation. Since there are no steps such as specifically binding carriers with magnetic properties to nucleic acids, and a preliminary preparation step in the prior art, in the kit content of the present invention, for example, the number of pipetting and the number of chemicals put into the sample are reduced. By means of the present invention, the risk of contamination is also reduced due to the reduction of pipetting and chemical use. By means of minimizing the number of steps and reducing the risk of contamination in the present invention, a safer isolation kit is provided than manual isolation in the prior art.

Another object of the present invention is to provide a kit with low cost to produce and implement, that contains few materials in its production, and that allows nucleic acid isolation with reusable/replaceable elements. Said kit comprises a modular and reusable magnetic gradient patch at the bottom part, a mold in which this gradient patch is placed, and a tube with flat-bottom, made of glass or its derivatives positioned on said patch. Chemicals and magnetic elements in the kits of the previous art cannot be used repeatedly since they are in direct contact with the samples, and this creates a cost disadvantage. In the present invention, The magnetic gradient patch can be used repeatedly, thereby providing a cost-effective kit by preventing the magnetic element from coming into contact with the sample.

In order for the kit subject to the present invention to be reused, it is sufficient to replace the tube positioned on the magnetic gradient patch. The mold in which the reusable magnetic gradient patch is placed, positioned under the flat bottom tube, is reusable; however, the glass tube must be replaced for accreditation of results due to the risk of contamination. In addition, a less costly kit is produced by means of the present invention since the production of prior art magnetic carriers is more costly than magnetic gradient patches. In addition, since there are no biological materials such as antibodies, etc. that may degrade in the content of the kit, which is the subject of the present invention, the problem that the kit cannot be used due to its deterioration is prevented.

Another object of the present invention is to provide nucleic acid isolation in a short time and, accordingly, to ensure that studies that require the use of isolated nucleic acids are concluded in a short time. The kit, which is the subject of the present invention, can be used to isolate RNA and DNA from body fluid to be used in the diagnosis of diseases transmitted through body fluid (blood, saliva, urine, mucous membranes, etc.).

By means of the present invention, a kit is provided that do not require long-term and costly preliminary preparation steps for target nucleic acid retention, that comprises a small number of process steps, have a low risk of contamination, that do not require direct binding and cleaning of magnetic elements to nucleic acids, that have elements that can be used over and over again, that accelerate and facilitate the nucleic acid isolation step and thereby accelerating the process and provide advantages in studies that require the use of nucleic acids due to the above mentioned disadvantages.

### DESCRIPTION OF THE FIGURES

FIG. 1: View of the parts that forms the kit.

FIG. 2: View of nucleic acid isolation process steps by the kit

### DESCRIPTION OF ELEMENTS/PARTS OF THE INVENTION

The parts and components in the figures are enumerated for a better explanation of the nucleic acid isolation kit of the present invention, and correspondence of every number is given below:
1: Tube
2: Magnetic gradient patch
3: Mold
A: Biological material for nucleic acid isolation
B: Nucleic acid isolation kit
C: Isolated nucleic acids

### DETAILED DESCRIPTION OF THE INVENTION

The present invention relates to a magnetic gradient-based nucleic acid isolation kit that provides nucleic acid (RNA/DNA) isolation from biological materials. The biological material mentioned herein can be body fluid, saliva, mucous membrane, blood, urine, sample containing viruses, or sample containing bacteria.

The kit, which is the subject of the invention, in its content, comprises;
a flat bottom tube (1) made of glass or its derivatives, configured to contain biological material,
a modular and reusable magnetic gradient patch (2) that is made of ferromagnetic metal powder and polymer, that is positioned outside and below the tube such that it is not in direct contact with the biological material to be placed in the tube (1), that is in direct contact with the entire flat bottom of the tube (1),
a mold (3) positioned under the flat-bottom tube (1), configured such that the magnetic gradient patch (1) is put therein

Said magnetic gradient patch (2) is a structure consisting of solid and dry ferromagnetic metal powder, preferably neodymium, iron, and boron alloy (Nd—Fe—B) powder in the form of washers trapped in a polymer material. The magnetic gradient patch (2) is reusable, and is magnetized by removing it from the mold (3) in case its magnetic effect decreases; and is then placed back into the mold (3) and under the tube (1). The entire flat bottom of the tube is in direct contact with the magnetic gradient patch. By means of the flat bottom of the tube (1) in the kit, the surface area where the magnetic gradient patch contacts the tube is enlarged. The bottom thickness of the flat bottom tube (1) is between 0.1 mm and 1 mm, and by means of this thinner bottom compared to the prior art, the interaction of the biological material and the magnetic gradient patch (2) is increased.

As a result of powdering the metals and combining the metal powder with the polymer, the magnetic gradient patch (2) is formed. The metal powder/polymer ratio in the magnetic gradient patch (2) is between 0.001-10% by weight. While preparing the magnetic gradient patch (2), which is the subject of the present invention, magnetic gradients are increased by using powdered ferromagnetic metals. The magnetic gradients present in the metal powder are increased by means of adding ferromagnetic metal powder into the polymer and homogeneously dispersing it in the polymer. The gradient increases since the surface area of the metal powders trapped in the polymer decreases and its distribution/dispersion within the polymer increases.

Said magnetic gradient patch (2) is made of neodymium, iron, and boron alloy (Nd—Fe—B) and polydimethylsiloxane (PDMS) materials in a sample of the present invention. As ferromagnetic metal powders, at least one of the group containing neodymium-iron-boron (Nd—Fe—B) alloy, iron (Fe), nickel (Ni), alnico, steel, cobalt (Co), samarium cobalt (Sm—Co) alloy, samarium-iron-nitrogen (Sm—Fe—N) alloy, ferrite magnet, permalloy (Ni—Fe alloy), manganese-bismuth/gallium (Mn—Bi/Ga) alloy, iron-nitrogen (Fe—N, Fe₁₆N₂) alloy and iron-cobalt (Fe—Co) alloy is selected as ferromagnetic metal powder.

As polymer, at least one polymer selected from the group containing polydimethylsiloxane (PDMS), epoxy resin, hydrogel, SU-8 polyethylene, which is an epoxy-based negative photoresist, polypropylene, polyvinyl chloride, rubber, nylon, PVB, silicone, polystyrene, neoprene, and polyacrylonitrile. However, for the kit of the present invention, the magnetic gradient patch (2), which has optimum properties and high magnetic properties, contains Nd—Fe—B and PDMS. The metal powders used in the invention are prepared by ball milling device and no additional chemical modification is required for preparing the metal powders. The present invention offers more cost-effective solutions in nucleic acid isolation with its multiple-use elements. Said kit, which is the subject of the present invention, provides isolation without the need for any device or additional chemical.

The mold (3), in which the magnetic gradient patch (2) is placed, may include a lid that can be opened and closed to enable the magnetic gradient patch (2) to be removed/replaced as required. In order for the kit of the present invention to be reused, it is sufficient to replace the tube (1) positioned on the magnetic gradient patch (2). In case of a decrease in the magnetic property of the magnetic gradient patch (2), the kit can be used again by removing the magnetic gradient patch (2) from the mold (3), magnetizing it, and putting it back into the mold (3).

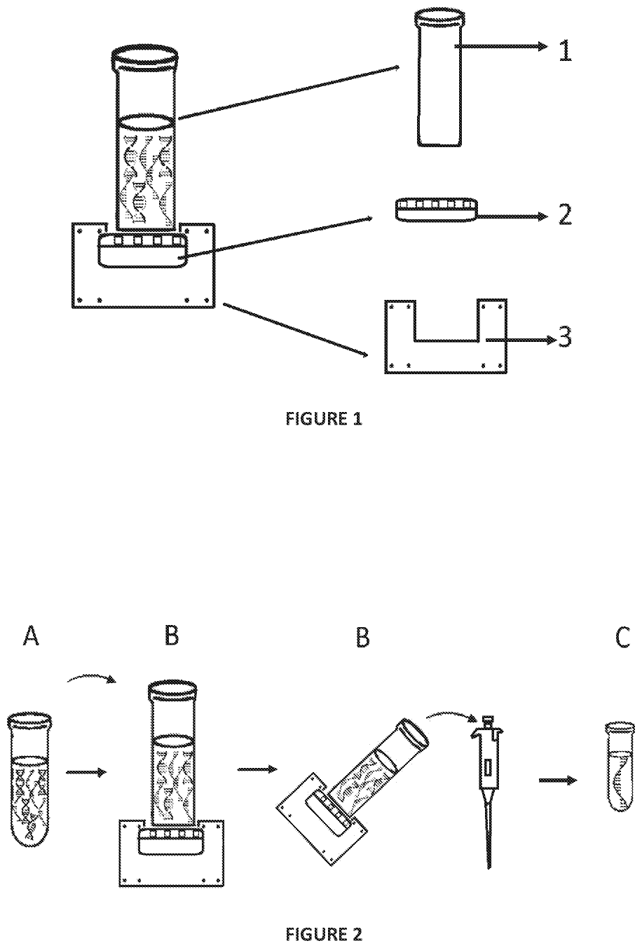

The modular/replaceable magnetic gradient patch at the bottom of the kit of the present invention and the modular/replaceable tube on this patch makes the kit reusable. As seen in FIG. 2, for the use of the kit, primarily biological material containing RNA or DNA to be isolated as nucleic acid (A) is transferred into the nucleic acid isolation kit (B), which consists of the tube (1) and magnetic gradient patch (2) according to the present invention, with the help of a micro pipette. As seen in FIGS. 1 and 2, the magnetic gradient patch (2) is positioned under the tube such that it is not in direct contact with the biological material/sample placed in the tube (1).

Both DNA and RNA are negatively charged due to the phosphate backbone and bonds formed between phosphorus atoms and oxygen atoms. As seen in FIG. 2, the nucleic acids in the biological material interact magnetically with the magnetic gradient patch (2) due to negative charges, and nucleic acid retention is ensured by the magnetic gradient patch (2) at the bottom of the tube (1). The nucleic acids remain at the bottom of the tube unless the magnetic gradient patch (2) is separated from the tube by the magnetic field effect. As shown in FIG. 2, the nucleic acids in the nucleic acid isolation kit (B) of the present invention accumulate at the bottom of the tube after a waiting period of 1-30 minutes since the magnetic gradient patch (1) is located at the bottom of the tube. Separation process according to the volume of biological material used can be performed by gently bending the tube (1) while there is a magnetic gradient patch (2) at the bottom of the tube, or by pulling out the part that is not involved (not needed) with the help of a micropipette. After removing the unnecessary portion, the magnetic gradient patch (2) is removed and the isolated nucleic acids remain at the bottom of the tube. Nucleic acids can be collected at this step by using an appropriate supplement solution. After removing the supernatant remaining in the tube (1), nucleic acids can be taken from the tube with a suitable washing solution; wash solutions used in nucleic acid isolation known in the art can optionally be used in this step. Isolated nucleic acids (C) remaining at the bottom of the tube are taken into a separate tube and separated for use in subsequent studies.

The production of the kit is easier and has a lower cost compared to the prior art since there is no chemical modification process and preliminary preparation step that can be considered complex in the production stage of the kit, which is the subject of the present invention. In an embodiment of the present invention, in the production stage of the kit, there are process steps of; preparing the PDMS/curing agent mixture, preparing Nd—Fe—B/PDMS patterned patch, and magnetizing the Nd—Fe—B/PDMS patch.

In an embodiment of the present invention, In the step of preparing the PDMS/curing agent mixture, PDMS, which is a member of the polymeric silicone group, is optically clear, non-toxic, easy to use and non-flammable is used. PDMS is mixed with the curing agent until being homogeneous, and the ratio of PDMS to Silicone elastomer curing agent is 10:1. In the preparation of Nd—Fe—B/PDMS patterned patch, flexible magnetic strips with (N, S, N, . . . ) magnetic poles are used. With the use of these lines, a patterned distribution of Nd—Fe—B powders is achieved. Fully dried metal powders are poured into a petri dish placed on magnetic strips (Magnetic strips with regular distribution as N, S, N, S . . . ) aligned at a 90 degree angle in order to ensure uniform distribution of Nd—Fe—B powders in the polymer. Then, the PDMS/curing agent mixture is poured onto the patterned Nd—Fe—B powders and dried on a hot plate at 75° C. for ˜60 minutes. Then, in the step of magnetizing the Nd—Fe—B/PDMS patch, the patch is magnetized in a 2.5 T magnetic field with a C-frame electromagnet in order to produce a magnetic gradient on top of the Nd—Fe—B/PDMS patch, and thereby obtaining magnetic gradient patch (2).

In an embodiment of the present invention, A study was conducted with the SARS-CoV-2 positive control material, which is one of the samples that can be used with the present invention to test the retention of a medical sample or nucleic acid with the magnetic gradient patch (2). The negatively charged SARS-CoV-2 positive control material was introduced into the isolation kit with the help of a micro pipette under the magnetic gradient created by the magnetic gradient patch (2), preferably the patch containing Nd—Fe—B, in the kit, which is the subject of the present invention. After a waiting period of 1-30 minutes, the upper liquid, which did not contain nucleic acid, was discarded, the sample collected at the bottom of the tube by magnetic gradient was drawn with a pipette, and the withdrawn liquid was subjected to RT-PCR analysis. Consequently, it was observed that the RNA positive control material, which was separated from the biological material with the isolation kit of the present invention, was retained in the kit.

In cases where separate RNA and DNA separation is required from the sample containing nucleic acid isolated by the method of the present invention, in order to separate RNA and DNA from each other, chloroform and phenol can be added to the sample after isolation with the kit. Apart from this technique found in the previous art and used very frequently, RNA and DNA separation can be performed in the same sample with salt precipitation or ethanol precipitation methods. However, these chemical processes are not within the scope of the kit of the present invention and are optional, and in fact, nucleic acid isolation with the present invention is provided only by magnetic gradient patch (2). Different purification steps can be applied after the isolation process, that is, the separation of nucleic acid from biological material, is completed with the kit that is the subject of the present invention, depending on preference and in which experiment the isolated nucleic acid will be used. Nucleic acids isolated directly with the kit according to the invention without applying these purification steps, are used in RT-PCR for the detection of SARS-CoV-2 performed in an embodiment of the above described invention, and give definitive results. By means of the kit, which is the subject of the present invention, nucleic acid separation from biological materials is provided only based on the magnetic gradient principle.

1. A kit for nucleic acid isolation from biological material without the use of any chemicals, characterized by comprising;
a flat bottom tube (1) made of glass or its derivatives, configured to contain biological material,
a modular and reusable magnetic gradient patch (2) that is made of ferromagnetic metal powder and polymer, that is positioned outside and below the tube such that it is not in direct contact with the biological material to be placed in the tube (1), that is in direct contact with the entire flat bottom of the tube (1),
a mold (3) positioned under the flat-bottom tube (1), configured such that the magnetic gradient patch (1) is put therein.

2. A kit according to claim 1, characterized in that, at least one of the group comprising polydimethylsiloxane (PDMS), epoxy resin, hydrogel, SU-8 polyethylene, which is an epoxy-based negative photoresist, polypropylene, polyvinyl chloride, rubber, nylon, PVB, silicone, polystyrene, neoprene, and polyacrylonitrile is selected as said polymer.

3. A kit according to claim 1, characterized in that, at least one of the group comprising neodymium-iron-boron (Nd—Fe—B) alloy, iron (Fe), nickel (Ni), alnico, steel, cobalt (Co), samarium cobalt (Sm—Co) alloy, samarium-iron-nitrogen (Sm—Fe—N) alloy, ferrite magnet, permalloy (Ni—Fe alloy), manganese-bismuth/gallium (Mn—Bi/Ga) alloy, iron-nitrogen (Fe—N) alloy and iron-cobalt (Fe—Co) alloy is selected as said ferromagnetic metal powder.

4. A kit according to claim 1, characterized in that, said flat bottom tube (1) has a bottom thickness between 0.1 mm and 1 mm in order to increase the interaction of the biological material and the magnetic gradient patch (2).

5. A kit according to claim 1, characterized in that, the metal powder/polymer ratio in said magnetic gradient patch (2) is between 0.001-10% by weight.

6. A kit according to claim 1, characterized in that, said biological material is body fluid, saliva, mucous membrane, blood, urine, sample containing virus or sample containing bacteria.
